(19) United States
(12) Patent Application Publication
(10) Pub. No.: US 2006/0257492 A1
(43) Pub. Date: Nov. 16, 2006

(54) Suspension of calcium phosphate particulates for local delivery of therapeutic agents

(75) Inventors: Hai Bo Wen, Warsaw, IN (US); Panjian Li, Fort Wayne, IN (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Products, Inc.

(21) Appl. No.: 11/129,246

(22) Filed: May 13, 2005

(51) Int. Cl. A61K 38/18; A61K 9/14; A61K 33/42

(52) U.S. Cl. 424/489; 424/602; 514/12; 977/906

(57) ABSTRACT

Disclosed herein are methods for preparing and using porous, crystalline biomimetic bioactive compositions of calcium phosphate with at least one therapeutic agent. The bioactive composition has strong adsorption properties for therapeutic agents which adsorb to the calcium phosphate with a high affinity. The bioactive composition also provides a sustained release implant that can be used for localized delivery of therapeutic agents. This localized delivery of therapeutic agents, promotes repair, healing, or regeneration of hard and soft tissues.

### FIELD OF THE INVENTION

The invention relates to bioactive compositions for localized delivery of a therapeutic agent to a region in a subject.

### BACKGROUND OF THE INVENTION

Orthobiologics is an emerging research field that is transforming the clinical focus of othopedics from traditional implants or devices to biologically based products for hard and soft tissue regeneration. Orthopedic and dental implants have been playing a critical role in the reconstruction of total knee and hip joints, spine, teeth/root systems, and in the repair of large bony defects; and in bone fracture fixation and healing as well. However, implant loosening, post-surgical infection, fracture nonunion and unpredictable periodontal regeneration are still issues of concern.

Bone and tissue formation has been encouraged by adding biological and therapeutic agents to an implantable substrate. However, many of these agents have to be properly incorporated into or onto the substrate in order to be clinically efficacious. A major problem for the clinical use of these agents is an appropriate delivery system. An ideal delivery system is one that is capable of maintaining an agent in situ for sufficient time for it to interact with target cells and for the agent to be released at an effective, but safe concentration during tissue repair/healing. In many instances this is only possible through the use of higher than necessary concentrations of the agent. However, some agents, such as bone morphogenetic protein (BMP), may cause serious side effects if overdosed locally or systematically.

Accordingly, there remains a need for methods and compositions that effectively deliver therapeutic agents to a target site to improve bone and soft tissue regeneration.

### SUMMARY OF THE INVENTION

The present invention provides methods for preparing and using crystalline, porous biomimetic bioactive compositions of calcium phosphate particles with at least one therapeutic agent. The bioactive composition has strong adsorption properties for therapeutic agents which adsorb to the calcium phosphate with a high affinity. The bioactive composition also provides a sustained release implant that can be used for localized delivery of therapeutic agents. This localized delivery of therapeutic agents promotes repair, healing, or regeneration of hard and soft tissues. The bioactive composition may also be used for localized delivery of therapeutic agents effective to treat diseases, including cancer and osteoporosis. In addition, the bioactive compositions may be used to locally deliver nucleic acid molecules for gene therapy methods. The nucleic acid molecule can be used to overexpress an encoded protein at the target region, or to silence gene expression at the target region.

The localized delivery of the therapeutic agent using the methods and compositions of the invention has certain advantages as it avoids the adverse effects of systematic administration by locally providing the desired concentration of the therapeutic agent at the target site. Hence, the need to systemically deliver high concentrations of a therapeutic agent is avoided.

Accordingly, in one aspect, the invention pertains to a bioactive composition for localized delivery of a therapeutic agent. The composition comprises a suspension of calcium phosphate particles, such as porous, crystalline, biomimetic apatite particles, and at least one therapeutic agent that is incorporated within the particles. Other examples of calcium phosphate particles that can be used are amorphous calcium phosphate, monocalcium phosphate monohydrate, monocalcium phosphate anhydrous, dicalcium phosphate dehydrate, dicalcium anhydrous, octacalcium phosphate, apatite, hydroxyapatite, tricalcium phosphate, and mixtures thereof. In one aspect, the composition is an injectable suspension and the particles are sufficiently small that they can be injected. The calcium phosphate particle can also be biodegradable. The composition of the invention is also biocompatible and mimics the inorganic composition of blood plasma.

In one embodiment, the bioactive composition can be delivered to the target site as an injectable suspension. In another embodiment, the bioactive compositions can be formulated in a variety of ways, e.g., a gel, a paste, and the like, and delivered to the target site. In one embodiment, the bioactive composition is formulated into a gelling agent such as a hydrogel. The gelling agent can be an injectable hydrogel that solidifies in situ.

In another aspect, the invention provides a method of preparing an injectable formulation of a bioactive composition by providing a calcifying fluid comprising calcium and phosphate ions. The calcium and phosphate ions are precipitated as calcium phosphate from the calcifying fluid. The precipitated calcium phosphate can be separated from the calcifying fluid and suspended in a suspending fluid. The suspended precipitate can be mechanically agitated, e.g., by sonication, to produce biomimetic calcium phosphate particles, having a particle size sufficiently small to be formed into an injectable suspension.

At least one therapeutic agent can be added to the calcium phosphate particles. The therapeutic agent can be added to the calcifying fluid before precipitation such that upon precipitation the therapeutic agent is complexed with the calcium phosphate and precipitated along with the calcium phosphate. Alternatively, the therapeutic agent can be added before, during, or after mechanical agitation, depending on the nature and stability of the therapeutic agent.

In another embodiment, the invention pertains to a bioactive composition comprising porous, crystalline biomimetic apatite particles made by mechanically agitating pre-made calcium phosphate particulates. At least one therapeutic agent can be added before, during, or after mechanical agitation.

In yet another aspect, the invention pertains to a method of delivering a therapeutic agent to a localized region in a subject by providing a bioactive composition comprising porous, crystalline biomimetic apatite particles with at least one therapeutic agent incorporated therein. This bioactive composition can be delivered to a region of a subject where the therapeutic agent is to be active, such that the therapeutic agent is able to elute from the composition.

### BRIEF DESCRIPTION OF DRAWINGS

FIG. 1A is an image of the characteristic morphology of calcium phosphate minerals formed in the early stage of biomimetic coating process;

FIG. 1B is an image of the characteristic morphology of calcium phosphate minerals formed in the end stage of biomimetic coating process;

FIG. 2A is a chart of showing XRD diffraction peaks of biomimietic apatite and a hydroxyapatite (HA) reference scan;

FIG. 2B is an FTIR spectrum of the biomimetic apatite powder indicating that the powder contains carbonate content, similar to bone mineral;

FIG. 3A is an ESEM micrograph of particulates collected from the biomimetic apatite suspension;

FIG. 3B is an FTIR scan demonstrating that the composition of the particulates is carbonated apatite;

FIG. 4 is a flow chart showing the steps of a protein absorption test of the biomimetic apatite powder;

FIG. 5 is a HPLC loading profile of osteocalcin;

FIG. 6 is a HPLC elution profile of osteocalcin;

FIG. 7 is a HPLC profile of osteocalcin after being released from the biomimetic apatite minerals; and

FIG. 8 is a HPLC loading profile of TGF-β1 in a sodium acetate buffer.

### DETAILED DESCRIPTION OF THE INVENTION

Certain exemplary embodiments of the invention will now be described to provide an overall understanding of the principles of the structure, function, manufacture, and use of the methods and compositions disclosed herein. Those skilled in the art will understand that the methods and compositions specifically described herein are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The invention provides methods and bioactive compositions for localized delivery of therapeutic agents using a suspension of porous, crystalline biomimetic particles with at least one therapeutic agent dispersed within and throughout the apatite particles. The particles of the suspension are sufficiently small that the particles and the therapeutic agent can be delivered to the subject by injection. The composition is one that is biocompatible and biomimetic in that it approximates the inorganic profile of blood plasma.

Features of the bioactive composition that make it useful for local delivery of a therapeutic agent are the porous nature and the small size of the calcium phosphate particles. These properties contribute to the availability of an increased surface area onto which the therapeutic agent is able to adsorb. This feature allows the therapeutic agent to be retained within the bioactive composition and released locally over time.

One skilled in the art will appreciate that the composition is bioactive in that it has a physiological and/or biological effect on a cell, tissue, organ, or other living structure. For example, the bioactive composition has the ability to support cell activity and the ability to be assimilated with natural bone by the activity of the cells. In particular, the bioactive composition can promote bone growth and healing by delivering a therapeutic agent to a target region in a subject.

As noted above, the a particle size of the biomimetic apatite should be sufficiently small that the composition can be delivered to a subject by injection. In one embodiment, the particle size is less than about 1 millimeter. In one embodiment, the particle size is less than about 100 micrometers. In another embodiment, the particle size is in the range of about 1-10 micrometers. In another embodiment, the particle size is in range of about 5-10,000 nanometers. More specifically, the particle size can be in the following ranges: about 20-50,000 nanometers, about 100-10,000 nanometers, and about 1,000-10,000 nanometers, as measured by conventional particle size measuring techniques such as scanning electron microscopy, or using a static or dynamic light scattering particle size analyzer. The pore size of the particle can also vary. For example, in one embodiment, the pore size is less than one micrometer. In another embodiment, the pore size is in the range of about 1-1000 nanometers. More specifically, the pore size can be in the following ranges: about 20-200 nanometers, about 40-160 nanometers, about 60-140 nanometers, about 80-120 nanometers, or about 100 nanometers.

The increased surface area and porosity of the bioactive composition improve the adsorption of a therapeutic agent to the surface of the calcium phosphate apatite particles. It will be appreciated that the adsorption characteristics will be dependent on a variety of factors such as particle size, pore size, and the therapeutic agent or combinations of therapeutic agents being used. Thus, the bioactive composition is one in which an amount of therapeutic agent has adsorbed to the calcium phosphate particles, and one that causes a localized therapeutic effect when a therapeutically effective amount of the therapeutic agent is desorbed from the bioactive composition at the target site in the subject. The increased porosity also results in a bioactive composition that releases the therapeutic agent, or a combination of therapeutic agents, in a controlled manner over a selected period of time. The controlled manner of release may depend upon the rate of cellular breakdown of the apatite. The controlled manner of release may also be affected by adding factors that alter the differentiation of cells, for example bone regenerating proteins that alter the production of chondrocytes and chondroblasts, which help to degrade the apatite particles at different rates. In one embodiment, the selected period of time ranges from about one hour to one week, several months, or many years depending on the disease or disorder being treated and the therapeutic agent being used. This controlled release over a period of time avoids the phenomena of “burst release” in which most of the therapeutic agent is lost at the target site at the beginning of implantation.

One skilled in the art will appreciate that a variety of therapeutic agents can be used depending upon the condition to be treated. Exemplary therapeutic agents include, but are not limited to, a growth factor, a protein, a peptide, an enzyme, an antibody, an antigen, a nucleic acid sequence (e.g., DNA and RNA), an agonist or an antagonist, a hormone, an anti-inflammatory agent, an anti-viral agent, an anti-bacterial agent, a cytokine, an oncogene, a tumor suppressor, a transmembrane receptor, a protein receptor, a serum protein, an adhesion molecule, a neurotransmitter, a morphogenetic protein, a differentiation factor, an analgesic, a matrix protein, a cell, and combinations thereof.

In one embodiment, the therapeutic agent is a growth factor selected from the group consisting of transforming growth factor-beta-1, vascular endothelial-derived growth factor, hepatocyte growth factor, platelet-derived growth factor, hematopoetic growth factor, heparin binding growth factor, peptide growth factor, basic fibroblast growth factor, acidic fibroblast growth factor, and combinations thereof.

In another embodiment, the therapeutic agent is a bone regenerative protein (BRP). Examples of bone regenerative proteins include, but are not limited to, bone morphogenetic proteins (BMP), such as BMP-2 for osteoinductive, osteoblast differentiation, and apotosis; BMP-3 (osteogenin) which is most abundant BMP in bone and inhibits osteogenesis; BMP-4 which is osteoinductive; BMP-5 which induces chondrogenesis; BMP-6 for osteoblast differentiation and chondrogenesis; BMP-7 (OP-1) which is osteoinductive; BMP-8 (OP-2) which is also osteoinductive; BMP-9; BMP-10; BMP-11; BMP-12 (GDF-7) which induces tendon-iliac tissue formation; BMP-13 (GDF-6) which induces tendon and ligament-like tissue formation; BMP-14 (GDF-5) for chondrogenesis, which enhances tendon healing and bone formation; and BMP-15. Other therapeutic agents include, but are not limited to, bone growth factors such as insulin-like growth factor, epidermal growth factor, fibroblast growth factor, vascular endothelial growth factor, TGF-beta, platelet-derived growth factor (PDGF), and tumor necrosis factor, growth and differentiation factor-5; cytokines such as IL-2 and IL-6; hormones, and combinations thereof.

In another embodiment, the therapeutic agents is an analgesic. Examples of analgesics include, but are not limited to, opioid analgesic agents such as cyclazocine, piperidine, piperazine, pyrrolidine, morphiceptin, meperidine, trifluadom, benzeneacetamine, diacylacetamide, benzomorphan, hydromorphone, oxymorphone, levophanol, methadone, meperidine, fentanyl, codeine, hydrocodone, oxycodone, propoxyphene, buprenorphine, butorphanol, pentazocine or nalbuphine, and muscarinic analgesics such as neostigmine, muscarinic receptor agonists (e.g., acetylcholine and synthetic choline esters, and cholinomimetic alkaloids, e.g., pilocarpine, muscarine, and arecoline), anticholinesterase agents and combinations thereof.

In another embodiment, the therapeutic agent is an antibiotic which includes, but is not limited to, cefamandole, tobramycin, vancomycin, penicillin, cephalosporin C, cephalexin, cefaclor, cefamandole, ciprofloxacin, bisphosphonates, chlortetracycline hydrochloride, chloramphenicol, oxytetracycline and combinations thereof.

In another embodiment, it may be desirable to incorporate genes for factors such as nerve growth factor (NGF) or muscle morphogenetic factor (MMP), TGF-beta superfamily, which includes BMPs, CDMP, and MP-52. In yet another embodiment, the therapeutic agent is a receptor. Examples of receptors include, but are not limited to, EPO Receptor, B Cell Receptor, Fas Receptor, IL-2 Receptor, T Cell Receptor. EGF Receptor, Insulin Receptor, and TNF Receptor.

In another embodiment, the therapeutic agent is selected from the group consisting of anti-coagulants, anti-inflammatory agents, anti-proliferative agents, immunosuppressant agents (e.g., FK506), glycosaminoglycans, collagen inhibitors. In other embodiments, the therapeutic agent can be osteoinductive materials, osteoconductive materials, organic molecules, nucleoproteins, polysaccharides, glycoproteins, lipoproteins, and synthetic and biologically engineered analogs thereof, as well as engineered or modified catalysts and diagnostic agents.

In yet another embodiment, a combination of two or more therapeutic agents may be used in the bioactive composition, such as a bone regenerating protein, an antibiotic or an anti-inflammatory agent. From a clinical sense, one of the major implications arising from a surgery is a need to control the post-operative inflammation or infection. An implant of the bioactive composition and an antibiotic reduces the chances of local infection at the surgery site, contributing to an infection-free environment, thus faster bone healing process. The anti-inflammatory agents control the degree of inflammation and also help in the bone healing process. The efficacy of antibiotics and anti-inflammatory agents is further enhanced by controlling their release from the bioactive composition by regulating the absorption and resorption rate such that they are delivered at their most effective dosage at the tissue repair site. Examples of anti-inflammatory agents include, but are not limited to ibuprophen. The antibiotics, anti-inflammatory agents, and bone regenerating proteins may be mixed within the bioactive composition, to locally deliver all or most of the necessary therapeutic agents to a localized region of a subject for bone tissue repair. It will be appreciated that any combination of the therapeutic agents can be used depending on the disease or disorder to be treated.

The bioactive composition may also contain additional additives such as a reinforcing material, a substrate or both. The additive can be selected based upon its compatibility with calcium phosphate and its ability to impart properties (e.g., biological, chemical or mechanical) to the composition, that are desirable for a particular therapeutic purpose. For example, the additives may be selected to improve tensile strength, alter elasticity, provide imaging capability, and/or alter flow properties and setting times of the bioactive composition. The additives are biocompatible, that is, there is no detrimental reaction induced by the material when introduced into the subject. In another embodiment, the bioactive composition may be formulated with a biocompatible substrate such as a hydrogel, as explained below.

One skilled in the art will also appreciate that additional components can be added to the composition to improve its efficacy. For example, the bioactive composition can be formulated with supplementary material in varying amounts and in a variety of physical forms, dependent upon the anticipated therapeutic use. The supplementary material may be in the form of sponges (porous structure), calcium phosphate coatings, meshes, films, fibers, gels, and filaments.

The bioactive composition can be made by precipitating calcium phosphate from a calcifying fluid that contains calcium and phosphate ions. The calcifying fluid closely mimics the inorganic composition of blood plasma. The concentration of calcium and phosphate ions in the calcifying fluid may vary and other ions such as magnesium and bicarbonate may also be present. One suitable example of a calcifying fluid is described in U.S. Pat. No. 6,569,489 to Li, which is incorporated herein by reference. The calcifying fluid of Li contains calcium ions, phosphate (HPO₄⁻²/H₂PO₄⁻¹) ions, bicarbonate (HCO₃⁻¹) ions and magnesium ions at a physiological temperature.

Calcium phosphate is insoluble at neutral pH, and it can be precipitated from the calcifying fluid by altering the pH of the fluid. The dissociation of bicarbonate (HCO₃⁻¹) from the calcifying fluid results in the production of carbon dioxide and hydroxyl ions, which in turn increase the pH of the calcifying fluid. The increase in pH results in precipitation of calcium phosphate from the calcifying fluid. In one embodiment, the pH of the calcifying fluid is in the range of about 3 to 13. In another embodiment, the pH of the calcifying fluid is in the range of about 5 to 8.

The time for precipitating the calcium phosphate from the calcifying fluid may vary, and it ranges from about one hour to one week. The time may also vary depending on factors such as whether a therapeutic agent is added to the calcifying fluid and the identity of the therapeutic agent. The temperature at which the calcium phosphate is precipitated can also vary within the range of about 4° C. to 95° C. In one embodiment, the precipitation of calcium phosphate is at temperature in the range of about 30° C. to 60° C., or in the range of about 37° C. to 45° C.

Once the calcium phosphate has precipitated, the precipitate is separated from the remaining calcifying fluid. This separation step can be performed in a cold room or at sub-ambient temperatures. Collection of the precipitate is carried out by any conventional means, including, but in no way limited to gravity filtration, vacuum filtration and centrifugation. The collected precipitate can be washed with distilled water or another physiologically suitable solution, and resuspended in suspending fluid such as distilled water, saline solution, and physiological buffer or medium such as phosphate buffered saline.

To alter the physical and dynamic properties of the calcium phosphate precipitate to render it suitable for injection and suitable as a carrier for a therapeutic agent, the precipitate can be subjected to mechanical agitation methods that reduce the size of the calcium phosphate particles into crystalline micrometer and nanometer sized particles. Examples of mechanical agitation methods include but are not limited to sonication, grinding, milling, and the like. An exemplary mechanical agitation method is sonication. A standard sonicator can be used such as the Sonic Dismemberator, Model 100, from Fischer Scientific. The power of the sonicator can range from about 100 Watts, 280 Watts, and 400 Watts. The power output can be adjustable, for example with some sonicators, the intensity of sonication can range from a scale of about 1-10, with a scale of 1 producing a 10% output of power, and a scale of 10 producing a 100% output of power. It will also be appreciated that the higher the power of the sonicator used for sonication, the larger the volume of buffer that can be used to suspend the particles. The frequency of sonication can range from about 1 kHz to about 40 kHz. The particles can be suspended in a suitable volume of buffer and sonicated for a desired period of time to reduce the size of the particles. The volume of buffer (e.g., PBS) can range from about 1-1000 ml, 1-500 ml, 1-200 ml, 1-100 ml, and 1-20 ml. The time of exposure to the sonicating frequency can range from about 1 minute to about 10 minutes. It will be appreciated that the particle size and crystal structure will vary depending on the time and frequency used for sonication.

Mechanical agitation produces calcium phosphate apatite particles with a particle size is less than about 1 millimeter. In one embodiment, the particle size is less than about 100 micrometers. In another embodiment, the particle size is in the range of about 1-10 micrometers. In another embodiment, the particle size is in range of about 5-10,000 nanometers. More specifically, the particle size can be in the following ranges: about 20-50,000 nanometers, about 100-10,000 nanometers, and about 1,000-10,000 nanometers, as measured by conventional particle size measuring techniques such as scanning electron microscopy, or using a static or dynamic light scattering particle size analyzer. The pore size of the particle can also vary. For example, in one embodiment, the pore size is less than one micrometer. In another embodiment, the pore size is in the range of about 1-1000 nanometers. More specifically, the pore size can be in the following ranges: about 20-200 nanometers, about 40-160 nanometers, about 60-140 nanometers, about 80-120 nanometers, or about 100 nanometers.

The crystalline structure of the calcium phosphate precipitate can be controlled by controlling physical parameters such as temperature, time, and pH during precipitation from the calcifying fluid. The control of these physical parameters may be important, for example, when a therapeutic agent is added to the calcifying fluid before precipitation.

When the therapeutic agent is added into the calcifying fluid before precipitation, both the calcium phosphate and the therapeutic agent are precipitated together. The therapeutic agent may also be added before, during, or after mechanical agitation of the calcium phosphate precipitate. It will be appreciated that the time at which the therapeutic agent is added will depend on the stability and nature of the therapeutic agent. For example, if the therapeutic agent is a protein, then precipitation at high temperature or pH may denature the protein and render it inactive. In such an instance, the protein may be added after the mechanical agitation step.

In another embodiment, the bioactive composition can be formed from a commercially available source of calcium phosphate particulates, such as those available from Sigma Chemicals. These particulates can be subjected to the same mechanical agitation methods described above and mixed with at least one therapeutic agent to produce the bioactive composition.

The bioactive composition can be used to deliver the therapeutic agent to a region in a subject. Thus, in one aspect, the invention pertains to localized delivery of at least one therapeutic agent to a region in a subject using the bioactive composition of the invention. The small size and porous nature of calcium phosphate particles in the bioactive composition lead to strong adsorption of therapeutic agents at high affinity, as well as the subsequent slow release of the therapeutic agent. The bioactive composition is characterized by its ability to interact with and adsorb therapeutic agents such as proteins, nucleic acids, and other substances, which make it an ideal delivery vehicle for the therapeutic agents to a target region in a subject.

The bioactive composition releases the therapeutic agent at the region of the subject in a controlled manner and eliminates the adverse effects arising from systemic administration of the therapeutic agent. In particular, use of the bioactive composition avoids “burst release” of the therapeutic agent and reduces the cost incurred by systematic administration of expensive therapeutic agents, such as growth factors. The methods and compositions of the invention also provide a minimally invasive technique for the treatment of difficult clinical situations, such as healing of large bone defects.

The delivery of the bioactive composition along with the controlled release of the therapeutic agent, primarily through desorption and elution activity, to a localized region in a subject, can be used to promote the natural bone remodeling process in that region. For example, the therapeutic agent can be a bone regenerative protein (BRP) which can be incorporated into the bioactive composition. BRPs increase the rate of bone growth and accelerate bone healing. Thus, an implant made of the bioactive composition and BRP would promote bone healing more rapidly than an implant without the BRP. The efficacy of the BRP is further enhanced by controlling the absorption and desorption of the BPR such that it is released at a controlled rate. This allows the BRP, calcium, and phosphorus to be delivered at the target site at the optimum dosage for bone growth.

Therefore, the bioactive composition may be used for orthopedic, maxillo-facial and dental applications and the bioactive composition can be fabricated to exist as a fine powder, pellets, three-dimensional shaped pieces, macroporous structures, thin films and coatings.

The bioactive compositions may also be used to deliver at least one therapeutic agent to a region of the subject that is not related to bone or cartilage repair and formation. Thus, for example, the bioactive composition may be used to deliver a therapeutic agent to a region with a tumor, e.g., a stomach tumor.

The bioactive composition can be delivered to the target region in the subject as a suspension or fluid by combining the bioactive composition with a suitable carrier such as water or other physiologically relevant fluid to produce a suspension that is injectable. Alternatively, the bioactive composition can be formulated with a biocompatible substrate and delivered to the subject. The biocompatible substrate must have properties that allow the therapeutic agent to remain active while within or on the substrate, as well as allowing the active therapeutic agent to diffuse, or elute out from the substrate.

In another embodiment, the bioactive composition can be formulated in a hydrogel and delivered in a hydrogel form to the target region. Hydrogels are polymers that absorb and swell in an aqueous environment. In one embodiment, the bioactive composition can be suspended in a hydrogel solution and injected directly into the target site where the hydrogel solidifies in situ. The solidified gel forms a matrix with the bioactive composition dispersed therein. In a further embodiment, the bioactive composition is suspended in a hydrogel solution which is poured or injected into a mold having a desired shape, then hardened to form a matrix having bioactive composition dispersed therein which can be implanted into the subject. Examples of materials which can be used to form a hydrogel include polysaccharides such as alginate, polyphosphazines, and polyacrylates, which are cross linked or block copolymers such as Pluronics™ or Tetronics™, polyethylene oxide-polypropylene glycol block copolymers which are cross-linked by temperature or pH, respectively.

In general, hydrogel polymers are at least partially soluble in aqueous solutions, such as water, buffered salt solutions, or aqueous alcohol solutions, that have charged side groups that can be cross linked. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Cations for cross-linking of the hydrogel polymers with acidic side groups to form a hydrogel are divalent and trivalent cations such as copper, calcium, aluminum, magnesium, strontium, barium, and tin, although di-, tri- or tetra-functional organic cations such as alkylammonium salts, e.g., R₃ N⁺ can also be used. Aqueous solutions of the salts of these cations are added to the polymers to form soft, highly swollen hydrogels and membranes. The higher the concentration of cation, or the higher the valence, the greater the degree of cross-linking of the polymer.

Anions for cross-linking of the hydrogel polymers to form a hydrogel are divalent and trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

Alginate can be ionically cross-linked with divalent cations, in water, at room temperature, to form a hydrogel matrix. Due to these mild conditions, alginate has been the most commonly used polymer for hybridoma cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

Polyphosphazenes are polymers with backbones consisting of nitrogen and phosphorous separated by alternating single and double bonds. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. The term bioerodible or biodegrable, as used herein, means a polymer that dissolves or degrades within a period that is acceptable in the desired application (usually in vivo therapy), less than about five years and most preferably less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes).

Several physical properties of the hydrogels are dependent upon hydrogel concentration. Increase in hydrogel concentration may change the hydrogel pore radius, morphology, or its permeability to different molecular weight proteins. Hydrogel pore radius determination can be effected by any suitable method, including hydraulic permeability determination using a graduated water column, transmission electron microscopy and sieving spheres of known radius through different agar gel concentrations (See, e.g., Griess et al., (1993) Biophysical J., 65:138-48). Other examples of hydrogels, include, but are not limited to, gelatin, collagen, agar, chitosan, and amelogenin.

One skilled in the art will appreciate that the volume or dimensions (length, width, and thickness) of the hydrogel comprising the bioactive composition can be selected based on the region or environment into which the hydrogel is to be implanted. For example, the hydrogel can have a length (defined by a first and second long end) of about 0.5 cm to about 5.0 cm, or about 10 cm to about 30 cm, and a width (defined by a first and second short end) of about 0.1 cm to about 1.0 cm, or about 2.0 cm to about 4.0 cm.

In another embodiment, the bioactive composition can be formulated into an injectable paste by mixing of the bioactive composition in an amount of water or physiologically compatible buffer sufficient to produce the desired consistency for injection. Most often this will be as thick as possible while still being able to pass through a 16-18 gauge syringe. Other gauged syringes may also be used such as a 12-14 gauge syringe. For some formulations requiring injection directly into solid tissue (e.g., into cortical bone of an osteoporosis patient), thinner consistencies (e.g., 1.5 ml H₂O/g bioactive composition) may be used.

In another embodiment, the bioactive composition can be formulated into a putty or past consistency, which can be introduced into the implant site. This putty is generally prepared by mixing the bioactive composition in an amount of water or physiological buffer sufficient to produce the desired consistency for manipulating the putty. Most often this will be as thick as possible while still being malleable by hand, although thinner more flowable consistencies may be desirable for many applications. The preferred consistency will be similar to that of clay. The hydrated material may be prepared immediately before use, or for several hours before use.

Application to the implant site will be performed according to the nature of the specific indication and the preferences of the surgeon. Similar considerations apply for cartilaginous implants as for bone. Injection techniques will be employed to deliver the bioactive composition directly into hard tissue (e.g., for osteoporosis patients) or into small fractures. For larger fractures putty-like consistencies can be used and will be implanted by hand or with a spatula or the like. Reconstruction will often use putty like forms but in some instances it will be more advantageous to pre-form, harden, and shape the material ex-vivo and implant a hardened form into the target region. Exposure or mixing of the bioactive composition with blood or body fluids is acceptable and may promote osteo- or chondrogenesis.

In another embodiment, the bioactive composition can also be applied as a thin film to a surface of an implant substrate by a variety of techniques. One exemplary technique is the dip-coating (C. J. Brinker et al., Fundamentals of Sol-Gel Dip Coating, Thin Solid Films, Vol. 201, No. 1, 97-108, 1991) which involves dipping the substrate in a suspension, withdrawing the substrate from the suspension at a constant speed, and drying the coated film at a temperature that does not destroy the therapeutic agent, for example at room temperature.

In spin-coating the suspension is dropped on a plate which is rotating at a speed sufficient to distribute the suspension uniformly by centrifugal action. Subsequent treatments are the same as those of dip coating. It is appreciated that there are a variety of other techniques which may be used to apply a thin film of the suspension to the substrate. Other techniques include a spraying of the suspension, roller application of the suspension, spreading of the suspension and painting of the suspension.

The following examples are illustrative of the principles and practice of this invention. Numerous additional embodiments within the scope and spirit of the invention will become apparent to those skilled in the art.

### EXAMPLES

### Example 1

### Preparation of Apatite from Calcifying Fluid

This example demonstrates how to prepare apatite from calcifying fluid described in U.S. Pat. No. 6,569,489, incorporated herein by reference. The calcifying fluid was prepared with analytical chemicals: NaCl, KCl, CaCl₂, K₂HPO₄, MgCl₂, NaSO₄, NaHCO₃ to achieve a close equivalence to the inorganic ion composition of blood plasma which are reported as follows: [Na⁺]=142 mM, [K⁺]=5.0 mM, [Ca²⁺]=2.5 mM, [Mg²⁺]=1.5 mM, [HCO₃⁻]=27.0 mM, [Cl⁻]=103.0 mM, [HPO₄²⁻]=1.0 mM, [SO₄²⁻]=0.5 mM.

In order to achieve a reasonable reaction rate and a good control of the coating process in vitro, adjustments can be made on some of the essential inorganic components of the blood plasma, such as Mg²⁺, a well-known inorganic inhibitor, Ca²⁺, HPO₄²⁻ and HCO₃⁻. One example is to prepare the solution with 3 mM Mg²⁺, 6 mM [HCO₃⁻] and [Ca²⁺] and [HPO₄²⁻] at a product of 15 mM². The reaction temperature was at 45° C. It took 3-4 days to make carbonated apatite minerals from one hundred liters of calcifying fluid. The duration of the reaction ranges from 1 hour to 1 week, depending on volume of the calcifying fluid, reaction temperature and pH, and crystallinity desired.

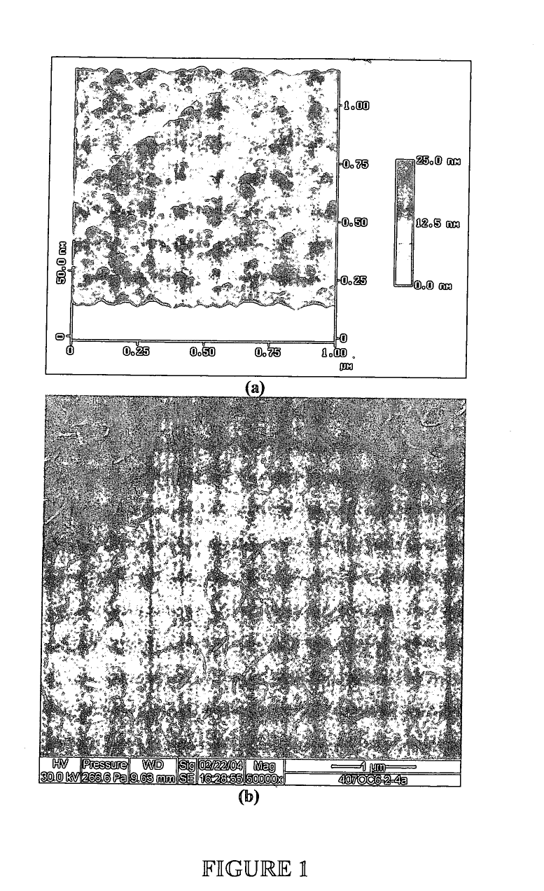

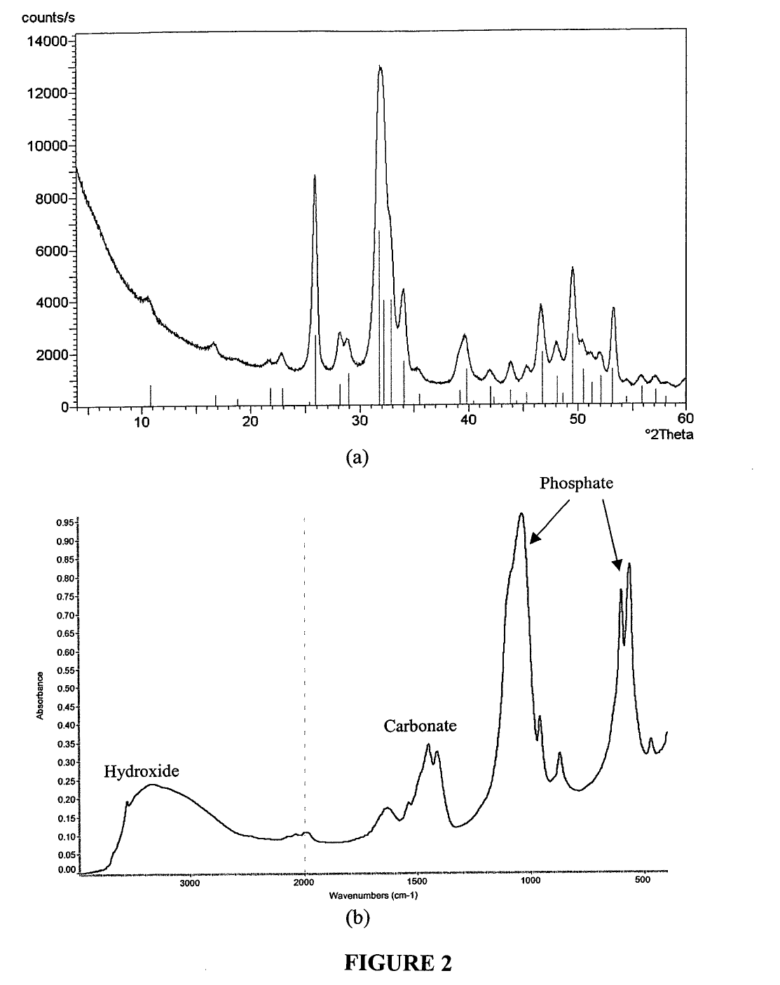

The precipitated calcium phosphate mineral formed in the calcifying fluid, on the walls of the reactor, and/or on the temporary substrates (glass or metal sheets), and can be collected for the preparation of powder or suspension. FIG. 1 shows the characteristic morphology of precipitated calcium phosphate minerals formed in the early stage (a, AFM) and in the end of a biomimetic mineralization process (B, SEM). The particulates formed in the early stage are nano-sized amorphous-like calcium phosphate particulates (FIG. 1A). These particulates are subsequently transformed into nanocrystalline carbonated-apatite with a unique nano-porous microstructure (FIG. 1B). The crystallographic structure and chemical composition of a biomimetic apatite powder prepared by this method is shown in FIG. 2. The prepared biomimetic powder was characterized by XRD and FTIR (FIG. 2). All the XRD diffraction peaks (FIG. 2A) can be assigned to HA reference scan (the HA was obtained from the Joint committee on Powder Diffraction International Centre (JCPDS) 09-0432, green). FTIR spectrum (FIG. 2B) indicated that the powder contains carbonate content, similar to bone mineral. These crystallites have a specific surface area over 100 m²/g and can adsorb over 3% weight protein from a bovine serum.

### Example 2

### Preparation of Apatite from Calcium Phosphate Particulates

Commercially available pre-made calcium phosphate particulates, or the calcium phosphate precipitate made by Example 1, was ground with mortar and pestle and suspended in 1-10 ml of phosphate buffered saline to a concentration of about 0.5 mg/ml to 1 mg/ml. The suspension was sonicated for 5 minutes at a frequency at an intensity scale of 1-10 using the Sonic Dismemberator, Model 100, from Fischer Scientific, to achieve smaller particulate size. The buffer/media can be made to mimic the inorganic composition of the aforementioned blood plasma. Proteins or other chemicals can be added for facilitating the conjugation of a particular therapeutic agent with mineral particulates and helping stabilize such conjugate and maintain the biological activity of the agent.

### Example 3

### Testing the Adsorption Properties of the Apatite

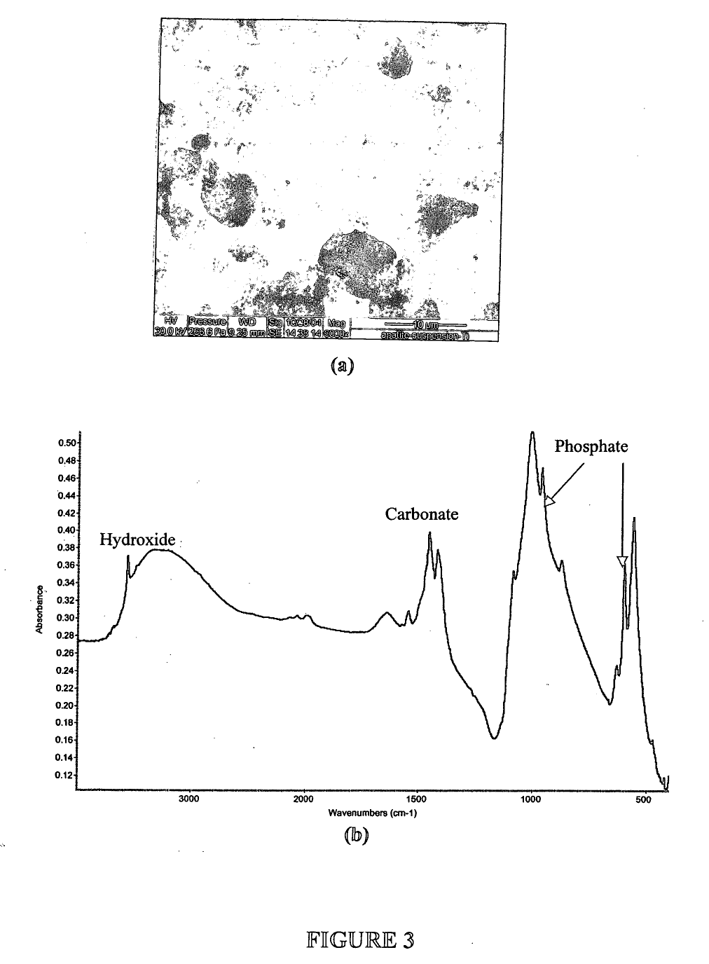

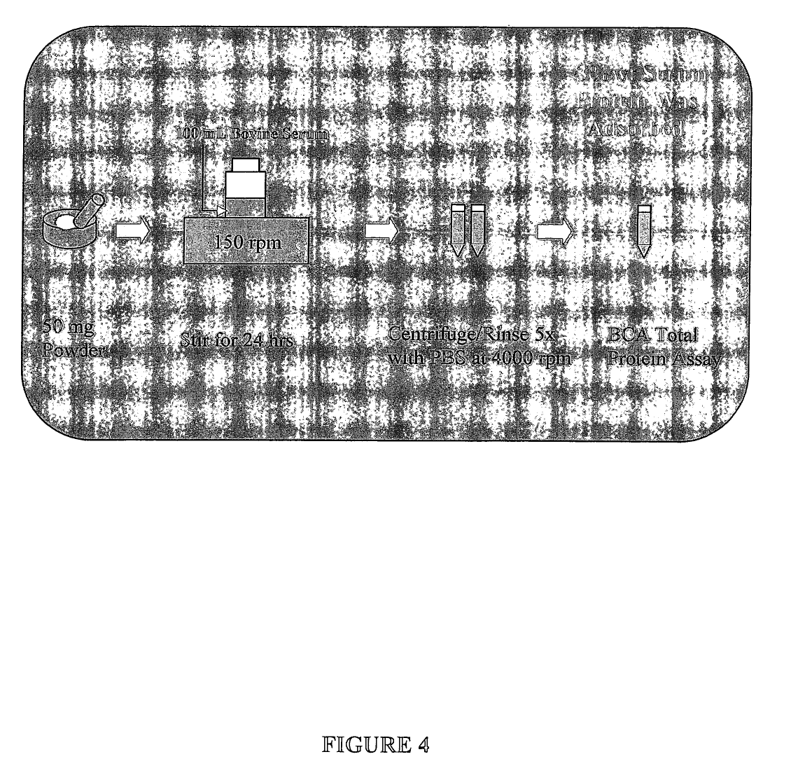

This example describes how to test the adsorption properties of the apatite using ESEM. A sample of the apatite prepared in Examples 1 and 2 was prepared for ESEM by placing a drop of the suspension on a Ti6Al4V coupon. The size of particulates in the suspension ranged from 1 to 10 μm as revealed by the ESEM micrograph in FIG. 3A. FTIR spectrum of the Ti6Al4V coupon (FIG. 3B) indicates that the composition of the particulates is carbonated apatite. As illustrated in FIG. 4, protein adsorption property of such powder was tested by incubating 50 mg ground powder with 100 ml of alpha calf fraction (bovine serum) on a rotating platform for 24 h. The powder was extensively rinsed with PBS by centrifuging (4000 rpm) and re-suspending for five times. The powder collected after the rinse was dissolved in 50 ml EDTA solution for BCA total protein assay. It was found that the powder retained more than 3% weight of serum protein even after the extensive rinsing process.

### Example 4

### Using the Apatite to Deliver Therapeutic Agents

A preliminary investigation on the feasibility of using biomimetic apatite suspension for delivering therapeutic agents was performed by employing two example proteins, i.e., osteocalcin, a bone tissue specific protein, and transforming growth factor-beta-1 (TGF-β1), one of the TGF-β superfamily proteins which includes BMP2, BMP7(OP-1) and GDF5.

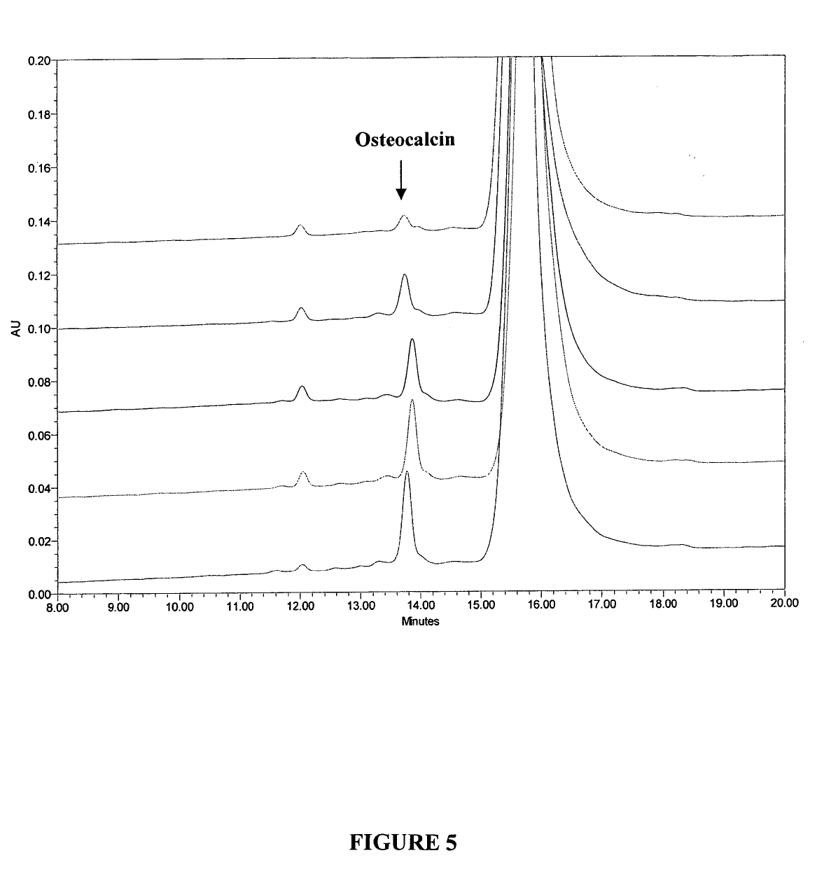

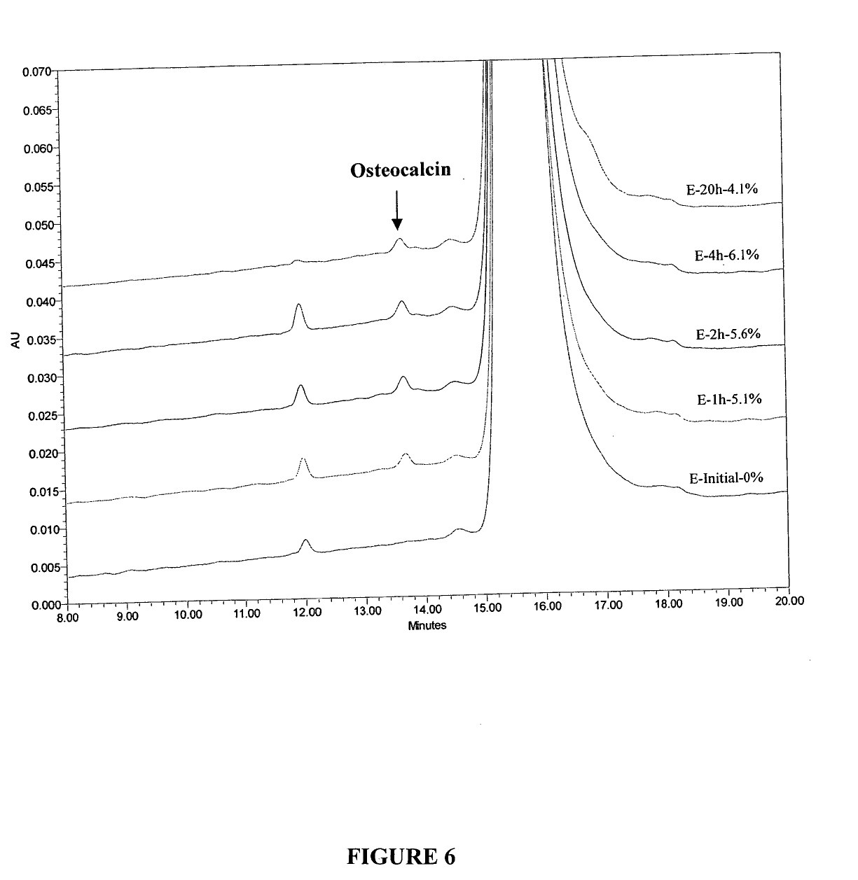

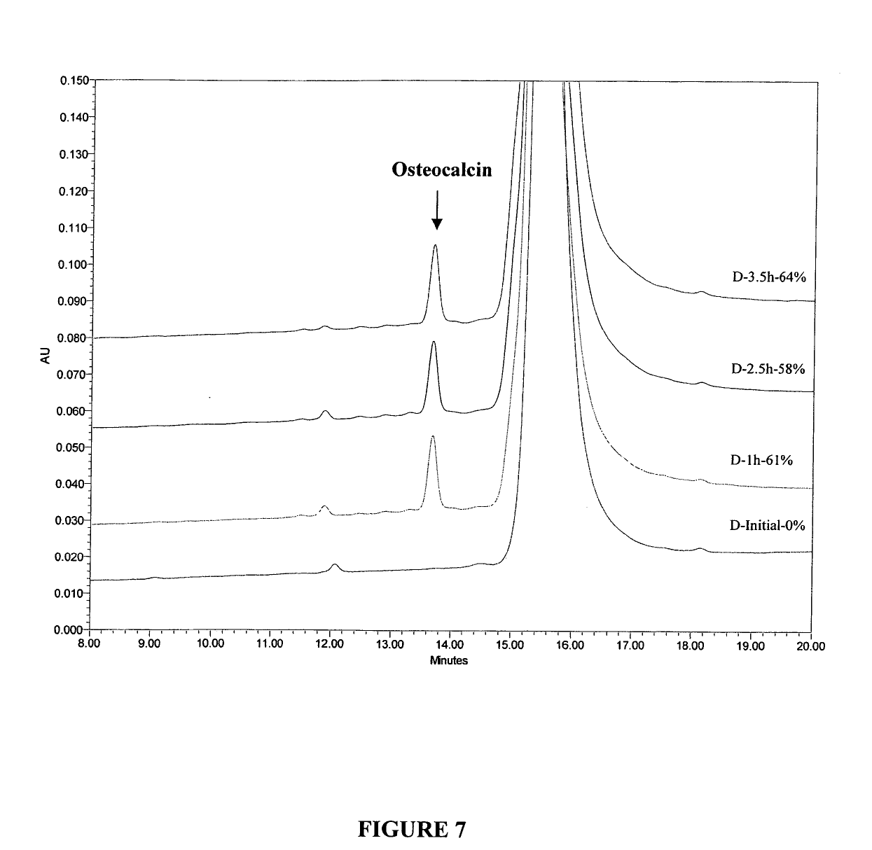

The test with osteocalcin was conducted by incubating biomimetic apatite prepared on Ti6Al4V substrates with phosphate buffered saline (PBS) containing 0.1 mg/mL (bovine serum albumin) BSA. FIG. 5 shows the high performance liquid chromatography (HPLC) elution profile of osteocalcin from the BSA solution after different incubation times. Eighty five percent of the original protein (20 μg) is adsorbed by biomimetic apatite crystals within 3 hours of test. An elution test was designed to further test the affinity of biomimetic apatite to osteocalcin in PBS containing 0.1 mg/mL BSA was measured (FIG. 6). Only ˜6% of the originally applied protein (20 μg) was eluted out from the biomimetic apatite crystals after 20 hours of incubation. Interestingly, the minerals have to be fully dissolved in acid in order to have the protein completely released from the minerals. FIG. 7 demonstrates that ˜64% of the protein (20 μg) was recovered after the dissolution.

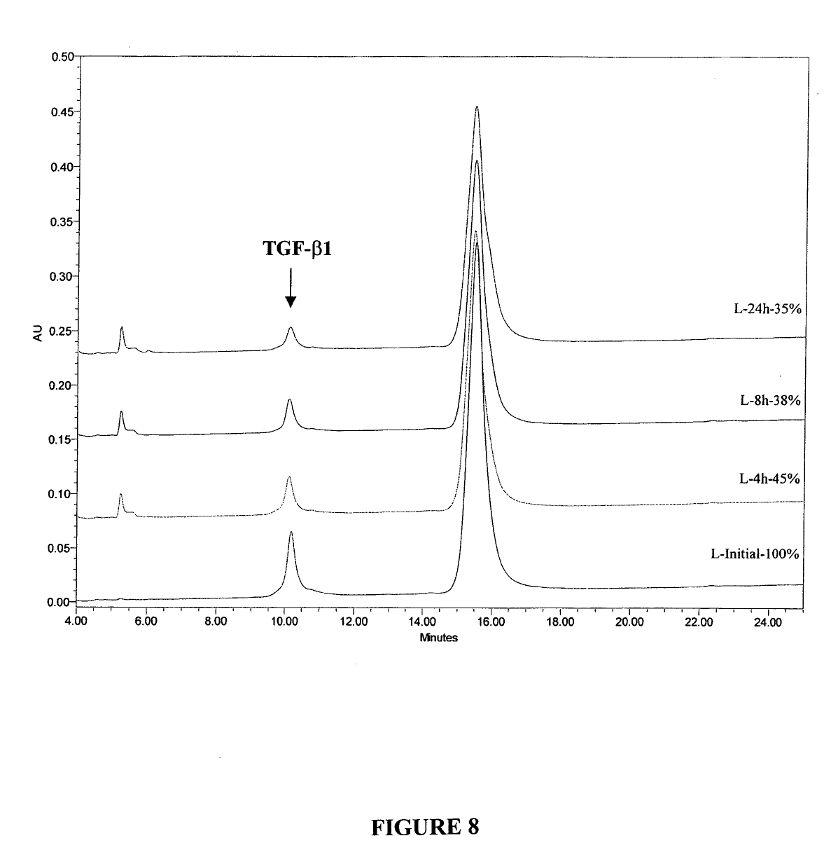

A parallel study was designed for testing the interaction between biomimetic apatite mineral with TGF-β1 by using a sodium acetate buffer containing 0.1 mg/mL BSA. FIG. 8 shows the HPLC elution profile of TGF-β1 from the incubating medium as a function of incubation time. Fifty five percent of growth factor (20 μg) was adsorbed by biomimetic apatite crystallites after 4 hours. Ten percent additional protein was adsorbed in the next 20 hour. The elution and dissolution profiles of TGF-β1 are very similar to those of osteocalcin.

These results demonstrate the controlled release of the protein without “burst release” of the absorbed protein. Thus, the suspension of biomimetic apatite particles offers a reliable, cost effective device for the delivery of therapeutic agents. The suspension is injectable and therefore minimally invasive. The size and crystallinity of the calcium phosphate particles can be tailored from nanometer to micrometer scale for different applications. The suspension can help deliver the therapeutic agents locally to the diseased sites, making it an attractive product for the treatment of other diseases, such as osteoporosis and cancer.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed:

1. A bioactive composition for localized delivery of a therapeutic agent, comprising
a suspension of calcium phosphate particles; and
a therapeutic agent incorporated on or within the particles.

2. The composition of claim 1, wherein the calcium phosphate particles are porous, crystalline biomimetic apatite particles and the therapeutic agent is incorporated on or within the biomimetic apatite particles.

3. The composition of claim 1, wherein the calcium phosphate particles are comprised of amorphous calcium phosphate, monocalcium phosphate monohydrate, monocalcium phosphate anhydrous, dicalcium phosphate dehydrate, dicalcium anhydrous, octacalcium phosphate, apatite, hydroxyapatite, tricalcium phosphate, and mixtures thereof.

4. The composition of claim 1, wherein the calcium phosphate particles have a particle size less than about one millimeter and a pore size less than about one micrometer.

5. The composition of claim 1, wherein the calcium phosphate particles have a particle size in the range of about 10-10,000 nanometers and a pore size in the range of about 1-1,000 nanometers.

6. The composition of claim 1, wherein the therapeutic agent is selected from the group consisting of a growth factor, a protein, a peptide, an enzyme, an antibody, an antigen, a nucleic acid sequence, an agonist, an antagonist, a hormone, an anti-inflammatory agent, an anti-viral agent, an anti-bacterial agent, a cytokine, an oncogene, a tumor suppressor, a transmembrane receptor, a protein receptor, a serum protein, an adhesion molecule, a neurotransmitter, a morphogenetic protein, a differentiation factor, an enzyme, a matrix protein, a cell, an analgesic, and mixtures thereof.

7. The composition of claim 6, wherein the growth factor is selected from the group consisting of transforming growth factor-beta-1, vascular endothelial-derived growth factor, hepatocyte growth factor, platelet-derived growth factor, hematopoetic growth factor, heparin binding growth factor, peptide growth factor, basic fibroblast growth factor, acidic fibroblast growth factor, and mixtures thereof.

8. The composition of claim 6, wherein the growth factor is transforming growth factor-beta-1.

9. The composition of claim 6, wherein the protein is selected from the group consisting of osteocalcin, bone morphogenetic protein, growth and differentiation factor-5, bone tissue specific protein, and cartilage derived morphogenetic protein.

10. The composition of claim 6, wherein the protein is bone tissue specific protein.

11. The composition of claim 1, wherein the bioactive composition has a physiologically biocompatible profile.

12. The composition of claim 1, wherein the bioactive composition is effective to retain or release the therapeutic agent over a period in the range of about one hour to several months.

13. The composition of claim 1, wherein the composition is in an injectable formulation.

14. The composition of claim 13, wherein the injectable formulation is an injectable gelling agent.

15. The composition of claim 14, wherein the gelling agent is selected from the group consisting of a hydrogel, an alginate, a gelatin, a collagen, a chitosan, an agar, an amelogenin, a polyphosphazine, and a polyacrylate.

16. The composition of claim 13, wherein the injectable formulation is a hydrogel.

17. The composition of claim 13, wherein the gelling agent forms a solid gel in situ.

18. The composition of claim 13, wherein the gelling agent forms a solid gel in vitro.

19. The composition of claim 13, wherein the suspension is applied to an implant.
